(19) United States
(12) Patent Application Publication
(10) Pub. No.: US 2019/0374379 A1
(43) Pub. Date: Dec. 12, 2019

(54) Portable hypothermia recovery pad

(71) Applicant: Dennis Harry Drew, Kenai, AK (US)

(72) Inventor: Dennis Harry Drew, Kenai, AK (US)

(21) Appl. No.: 15/998,006

(22) Filed: Jun. 9, 2018

(51) Int. Cl. A61F 7/08; A61F 7/00

(52) U.S. Cl. CPC A61F 7/08; A61F 7/007; A61F 2007/0087; A61F 2007/0082; A61F 2007/0024; A61F 7/0085

(57) ABSTRACT

This is a portable device that will safely stop the drop of the body core temperature of a hypothermic victim, and slowly raise the core temperature back to a safe level. This is accomplished by the use of a DC heating element, memory foam cushions, and a heat reflecting element. All of which is encased in a vinyl sleeve that is reusable.

### FIELD OF THE INVENTION

The present invention relates generally to medical devices. More specifically, the present invention is a portable device for raising the core body temperature of a hypothermia victim.

### BACKGROUND OF THE INVENTION

Hypothermia is a condition that sets in following prolonged exposure to a cold environment. The condition is characterized by a significant drop in the core body temperature of a victim. Symptoms in mild cases generally encompass shivering and hypertension while in severe cases, symptoms may include decreased heart rate, respiratory rate, and blood pressure. When attending to a hypothermia victim, it is of the utmost importance to raise the core body temperature to normal levels. A common practice is to wrap the victim in a blanket to warm his or her body. While this may be sufficient in cases of mild to moderate hypothermia, in more severe cases, this practice may lead to the condition worsening and even death. Wrapping a severe hypothermia victim's body causes the body to retain the cold, leading to the victim's death. It is also important to note that a hypothermia victim's core body temperature must not be raised too quickly as the shock of a sudden increase in temperature may cause the victim to experience a cardiac episode. The present invention seeks to address the aforementioned issues of hypothermia treatment methods and provide a more effective, safer, treatment device.

The present invention is a portable device for safely and effectively raising the core body temperature of a hypothermia victim. In its preferred embodiment, the present invention encompasses a housing sleeve that envelops a heating element. The heating element is sandwiched between two layers of memory foam and a single layer of heat reflective material. The layers of memory foam provide support for a victim's body as well as the internal components of the device. The memory foam conforms to the contours of a victim's body which prevents heat loss through any air gaps between the device and the victim's body. Heat generated by the heating element transmits through both layers of memory foam. The heat reflective material directs heat back towards the victim's body. In the preferred embodiment of the present invention, the heating element draws power from an external power source such as a 12-Volt battery. However, alternative embodiments of the present invention may utilize self-contained power sources or incorporate additional power sources.

The present invention is utilized by positioning the device in close proximity of a victim's spine and head by resting the victim against the device. This allows the device to most effectively raise the core body temperature of the victim. The heating element of the present invention increases temperature at a slow and steady rate. This allows the device to warm the victim's body without causing the victim to experience a sudden; shocking change in temperature. The present invention is portable and may be used wherever a power source is available. Additionally, the present invention does not require specialized training to use.

### BRIEF DESCRIPTION OF THE DRAWINGS

FIG. 1 is an isometric view of the housing sleeve of the present invention

FIG. 2 is an isometric view of the heating element of the present invention

FIG. 3 is an isometric view of the memory foam block of the present invention

FIG. 4 is an isometric view of the sheet of heat reflective material of the present invention

FIG. 5 is an isometric exploded view of the components of the present invention and an external battery power source

FIG. 6 is an isometric assembly view of the components of the present invention and an external battery power source

FIG. 7 is an internal isometric assembly view of the components of the present invention and an external battery power source

### DETAIL DESCRIPTIONS OF THE INVENTION

All illustrations of the drawings are for the purpose of describing selected versions of the present invention and are not intended to limit the scope of the present invention.

The present invention is a portable device that is used to raise the core body temperature of a hypothermia victim. In its preferred embodiment, the present invention comprises a housing sleeve, a heating element, two memory foam blocks, and a sheet of heat reflective material.

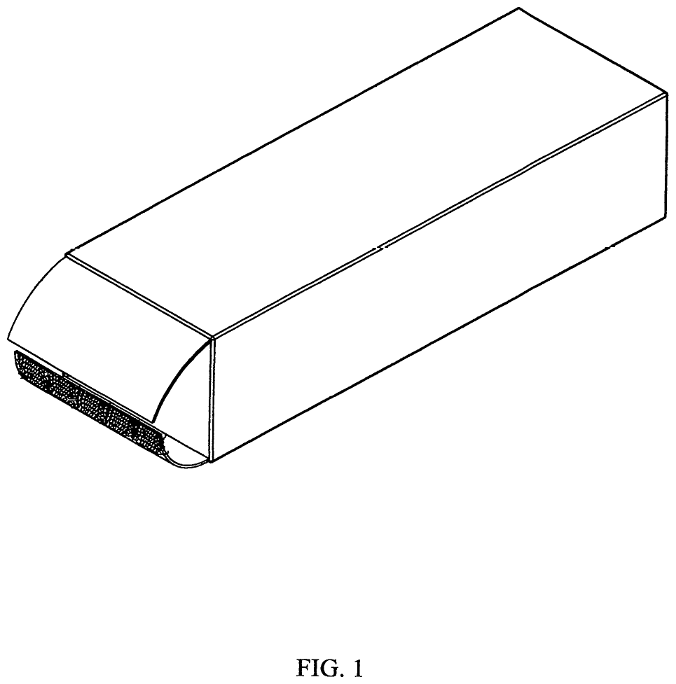

With respect to FIG. 1, in the preferred embodiment of the present invention, a housing sleeve comprises an approximately rectangular extrusion of material. The rectangular extrusion further comprises an empty space forming an interior hollow chamber. The interior hollow chamber is open to the exterior environment on one end of the rectangular extrusion. The open end of the rectangular extrusion may be closed from the exterior environment by means of two flaps that overlap and close the interior hollow chamber. In the preferred embodiment of the present invention, the closing mechanism utilizes Velcro hooks and loops present on the surfaces of the two flaps to close the interior hollow chamber from the exterior environment. The present invention is not limited with respect to material of the housing sleeve. In the preferred embodiment of the present invention, the housing sleeve is composed of a resilient material that may be quickly and conveniently cleaned, such as vinyl.

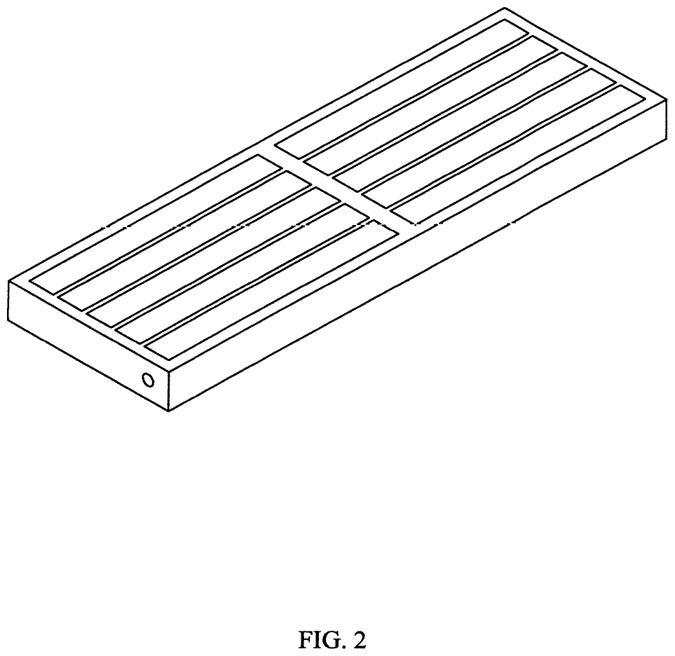

Referring to FIG. 2, in the preferred embodiment of the present invention, a heating element comprises a rectangular housing apparatus that encases an internal heater component. The present invention is not limited with respect to the type of heater utilized within the device. In the preferred embodiment of the present invention, the heating element is capable of generating heat up to a maximum temperature of 105° F. An integral feature of the present invention is the ability to reach this maximum temperature at a slow and steady pace in order to avoid shocking the victim with a sudden change in temperature. As such, any heating element utilized in the operation of the present invention must be capable of gradually increasing the victim's core body temperature. In the preferred embodiment of the present invention, the heating element further comprises a port for connecting to an external power source such as a 12-Volt battery. However, in alternative embodiments of the present invention, the heating element may incorporate a self-contained power source.

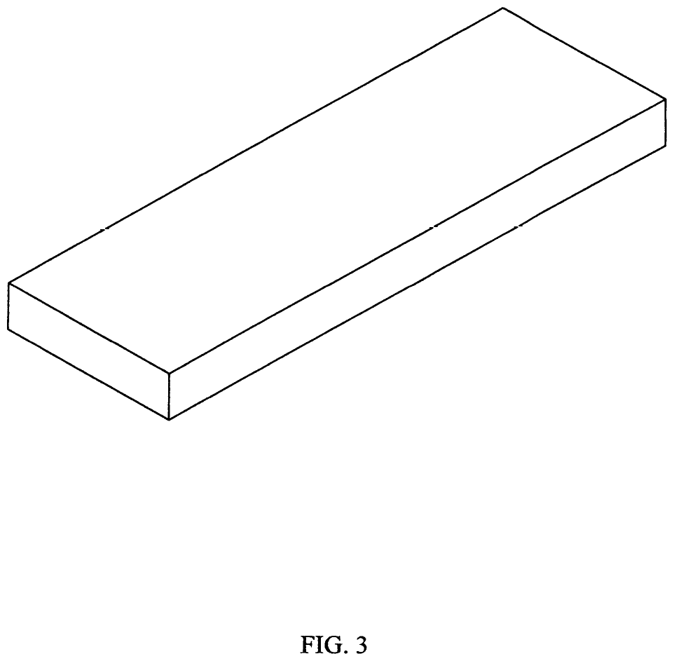

With reference to FIG. 3, in the preferred embodiment of the present invention, a memory foam block comprises a rectangular extrusion of polyurethane. The polyurethane material is compressible, flexible, lightweight, and resilient. It is important to note that the memory foam blocks must be soft enough that the freezing soft tissue of the hypothermia victim will not be damaged during the treatment process. Furthermore, the memory foam block must provide protection for the heating element of the present invention. The visco-elasticity of the polyurethane material causes the memory foam block to conform to the contours of a victim's body when the victim's body is placed against the memory foam block. Additionally, the polyurethane material has a relatively low thermal conductivity which results in a reduced rate of heat transfer through the material. This is beneficial as an object of the present invention is to raise a victim's core body temperature at a slow and steady rate in order to reduce the chance of shock and cardiac events. During normal use, the present invention incorporates two memory foam blocks.

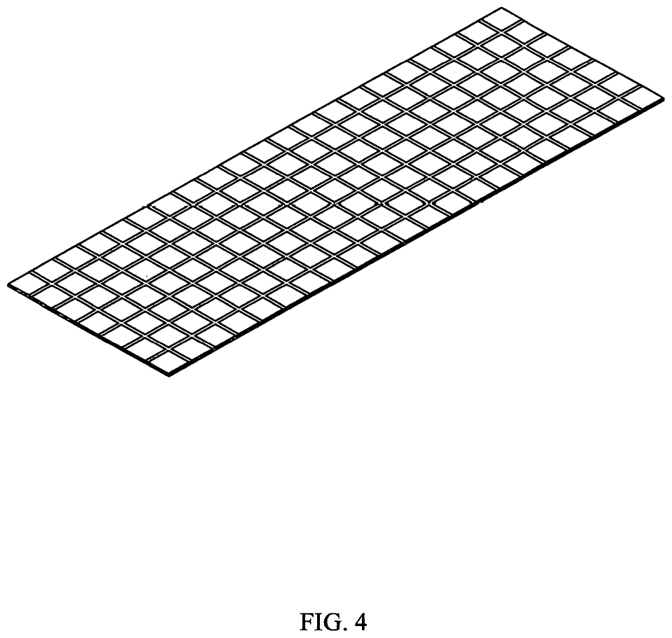

Regarding FIG. 4, in the preferred embodiment of the present invention, a sheet of reflective material comprises a flat, thin, and rectangular piece of foil. The material has high reflectivity and low emissivity and is capable of redirecting the heat generated by the heating element.

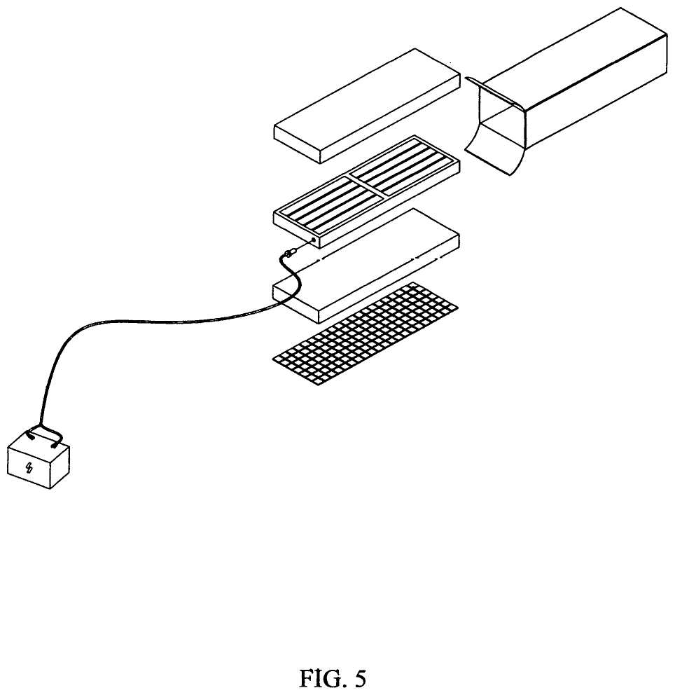

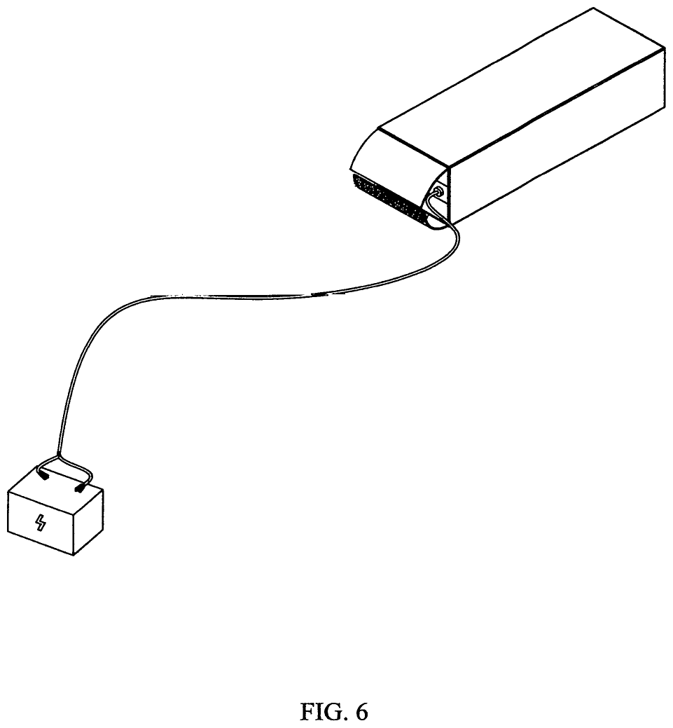

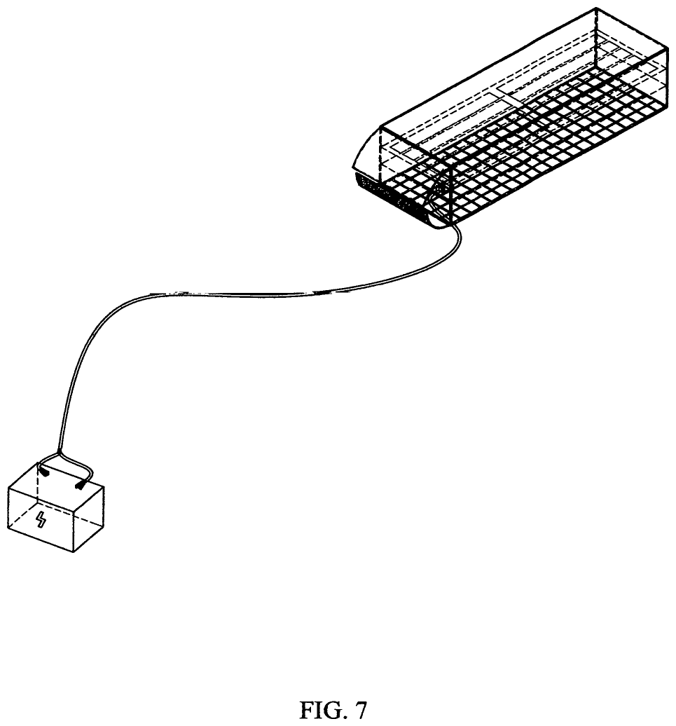

As shown in FIG. 5, the heating element is sandwiched between the two memory foam blocks and inserted into the interior hollow chamber of the housing sleeve. The sheet of reflective material is placed into the interior hollow chamber and is oriented such that the reflective sheet is opposite the side of the housing sleeve in contact with a victim's body. The heating element generates heat in the direction towards the victim's body and the direction away from the victim's body. The reflective sheet forces all heat transfer from the heating element to be oriented in the direction towards the victim's body for maximum efficiency. The housing sleeve completely encloses the heating element, memory foam blocks, and sheet of reflective material within its interior hollow chamber as can be seen in FIG. 6-7. Referring again to FIG. 6-7, the heating element of the present invention is connected to an external battery power source. However, as previously discussed, the present invention is capable of incorporating a self-contained power source into the heating element. This serves to increase portability of the present invention.

It is an object of the present invention to raise the core body temperature of a hypothermia victim. A further object of the present invention is to perform the aforementioned function in a safe and medically sound manner. The present invention is designed to slowly and steadily raise the core body temperature of a hypothermia victim in order to avoid the shock of a sudden change in temperature and a potential cardiac event. The heating element of the present invention gradually attains a maximum temperature of 105° F. It is a further object of the present invention to provide the aforementioned medical treatment while remaining portable. The present invention is portable as all internal components are encased within the housing sleeve of the present invention. Additionally, the present invention may be deployed wherever an external power source such as a 12-Volt battery is available. Alternative embodiments of the present invention in which the heating element incorporates a self-contained power source serve to further increase the portability of the present invention. It is an even further object of the present invention to provide efficient treatment without damaging the fragile soft tissue of the hypothermia victim. Efficient treatment is provided by the soft memory foam blocks that conform to the contours of the victim's body during the treatment process. The memory foam blocks prevent dissipation of heat from the heating element and maximize the heat transfer towards the body of the hypothermia victim. Additionally, the soft memory foam blocks provide support and cushioning for the victim's body during the treatment process. Finally, the present invention is designed in a manner such that treatment may be provided by non-trained users in addition to trained professionals. The present invention's mechanism of operation is straightforward to allow for non-trained users to provide treatment and save a hypothermia victim's life prior to the arrival of trained personnel.

Although the present invention has been explained in relation to its preferred embodiment, it is understood that many other possible modifications and variations can be made without departing from the spirit and scope of the present invention.

What is claimed is:

1. A portable hypothermia recovery pad comprising:
a vinyl sleeve that houses a DC heating element, two pieces of memory foam. and a heat reflecting foil sheet;

2. A portable heating device that was designed to heat slowly so as to not shock a hypothermic victim:

3. A portable heating device designed to conform to the curvature of the human spine.
